(19) United States
(12) Patent Application Publication
(10) Pub. No.: US 2022/0281808 A1
(43) Pub. Date: Sep. 8, 2022

(54) PREPARATION METHOD FOR DIPHENYLMETHANE DIISOCYANATE

(71) Applicant: Institute of Process Engineering, Chinese Academy of Sciences, Haidian District (CN)

(72) Inventors: Huiquan Li, Haidian District (CN); Liguo Wang, Haidian District (CN); Peng He, Haidian District (CN); Yan Cao, Haidian District (CN); Jiaqiang Chen, Haidian District (CN); Shuang Xu, Haidian District (CN)

(73) Assignee: Institute of Process Engineering, Chinese Academy of Sciences, Haidian District (CN)

(21) Appl. No.: 17/635,075

(22) PCT Filed: Aug. 17, 2020
(86) PCT No.: PCT/CN2020/109493

§ 371 Date: Feb. 14, 2022

(30) Foreign Application Priority Data
Aug. 15, 2019 (CN) 201910753037.4

(51) Int. Cl. C07C 263/04; C07C 265/14; C07C 265/12; B01J 23/72

(52) U.S. Cl. CPC C07C 263/04; C07C 265/14; C07C 265/12; B01J 23/72

(57) ABSTRACT

Disclosed is a preparation method for preparing diphenylmethane diisocyanate. The preparation method comprises: under a catalyst condition, performing a pyrolysis reaction on diphenylmethane dicarbamate in an inert solvent having a boiling point lower than that of diphenylmethane diisocyanate to obtain diphenylmethane diisocyanate.

### TECHNICAL FIELD

The present application relates to the technical field of organic chemical engineering, for example, a preparation method for diphenylmethane diisocyanate.

### BACKGROUND

Isocyanate is a general term for various esters of isocyanic acid which contains a functional group “—N═C═O” in molecular structure and is an important intermediate for organic synthesis. At present, the most widely used isocyanate in the industry is diphenylmethane diisocyanate (MDI), which mainly includes MDI 100 (4,4′-MDI), MDI 50 (a mixture of 50% 2,4′-MDI and 50% 4,4′-MDI) and polymeric MDI (PMDI), where PMDI is a mixture of 4,4′-MDI and polymethylene polyphenyl polyisocyanate. 4,4′-MDI is mainly used for the synthesis of spandex and elastomers, while PMDI is mainly used for the synthesis of polyurethane. Compared to 4,4′-MDI, PMDI has a larger market share (80%) and is the main raw material for preparing polyurethane due to its liquid state, easy transportation, good stability and performance more suitable for foaming and preparing polyurethane.

At present, the main method of producing MDI at home and abroad is a liquid phase phosgene method. In a process of liquid phase direct phosgenation, an amine compound is dissolved in a solvent, and phosgene is introduced into the solvent and reacted so that MDI is prepared. This process is particularly applicable to the amine compound with a high boiling point and low reactivity and is widely used in large-scale industrial production of isocyanate products such as MDI and toluene diisocyanate (TDI). Processes of the liquid phase phosgene method include a tank continuous process of liquid phase direct phosgenation (ICI) and represented by Huntsman, a tower continuous process represented by Bayer and Basf and a loop continuous process represented by Swedish International Chemical Corporation. Although the production process of phosgenation has been relatively mature, phosgene is easy to volatilize and a highly toxic gas, which causes serious potential safety hazards in the production process. Moreover, HCl, a by-product during the production of isocyanate by the phosgene method, corrodes a production device seriously so that a cost of the production is increased and the quality of the product is affected.

Therefore, with increasing environmental protection consciousness of people, a technical route of preparing isocyanate by a non-toxic and pollution-free green synthesis method has attracted more and more attention. Prior processes mainly include the liquid phase production of MDI through oxidization and carbonylation using aniline, carbon monoxide, ethanol and oxygen as raw materials, which is developed by Asahi Kasei Corporation of Japan; a route of producing MDI using aniline and CO₂ as raw materials, which is developed by Monsanto Company of the United States in the early 1990s; a process of producing MDI using nitrobenzene and carbon monoxide as raw materials, which is developed by Atlantic Richfield Company (ARCO) of the United States; a new non-phosgene process of producing MDI through oxidization and carbonylation of a mixture of nitrobenzene, aniline and methanol in the presence of a rhodium carbonyl complex or a ruthenium carbonyl complex as a catalyst, a process of replacing the phosgene with dimethyl carbonate (DMC) and other processes, which are jointly developed by Catalytica Associates/Halodor Topsoe and Koide Kokan Co., Ltd. of Japan. However, the above processes cannot be applied to industrial production due to complex devices and processes, harsh reaction conditions, low yields, high production costs and other reasons.

With increasingly high requirements for energy saving and environmental protection, researches on processes for non-phosgene preparation of isocyanate have emerged and been developed rapidly. Affected by the general environment, researchers propose a variety of non-phosgene preparation methods for isocyanate, among which there are three representative methods: a triphosgene (bis(trichloromethyl)carbonate, BTC) method, transesterification and carbamate pyrolysis. All of the above three methods avoid highly toxic phosgene, and use non-toxic raw materials and a green synthesis route, which meets the requirement for energy saving and environmental protection. Among the three methods, the carbamate pyrolysis has the most promising prospect of industrialization. The carbamate pyrolysis includes gas-phase pyrolysis and liquid-phase pyrolysis according to a reaction phase. In the gas-phase pyrolysis, a raw material (phenyl carbamate powder) is carried along with an inert gas into a gas reactor such as a fixed bed, a fluidized bed or the like and subjected to a pyrolysis reaction at a high temperature so that isocyanate is prepared. In the liquid-phase pyrolysis, the raw material (phenyl carbamate) and a solvent (with a high boiling point) are added at a certain ratio to a reactor and subjected to the pyrolysis reaction in the presence or absence of a catalyst under reduced pressure, normal pressure or increased pressure so that an isocyanate product is obtained.

Methyl diphenylmethane dicarbamate (MDC) is pyrolyzed using a solvent with a high boiling point so that MDI is prepared, which has the advantages of a fast reaction rate and easy separation. However, the process also has the following disadvantages: MDI at an outlet has a high concentration and is easy to polymerize, MDI and methanol are evaporated simultaneously so that a reversible side reaction is easy to occur, and after the reaction, the solvent with the high boiling point and a flocculent by-product are difficult to separate, resulting in the low reusability of the solvent.

CN1721060A uses the carbamate pyrolysis. In an inert solvent, an ultrafine metal oxide is used for catalyzing the pyrolysis of MDC at a reaction temperature of 150-300° C. so that MDI is obtained. However, the yield of MDI obtained through this reaction is only 52.1%-63.1%. In CN1850792A, MDC is pyrolyzed in an inert solvent such as dioctyl sebacate at a temperature of 210-290° C. under a pressure of 0.09-0.093 MPa so that MDI is obtained. MDC used for this reaction has a high concentration and a side reaction is easy to occur.

Therefore, it is an important subject of current researches to develop a method for preparing MDI with simple devices and processes, under mild reaction conditions and at a relatively high yield, so as to meet the requirement for industrial production.

### SUMMARY

An object of the present application is to provide a preparation method for diphenylmethane diisocyanate. The preparation method has a relatively high reaction conversion rate and yield, simple devices and processes and relatively mild reaction conditions, can meet the requirement for industrial production, and has a relatively high industrial application value.

To achieve the object, the present application adopts a technical solution described below.

In a first aspect, the present application provides a preparation method for diphenylmethane diisocyanate. The preparation method includes: in the presence of a catalyst, subjecting diphenylmethane dicarbamate to a pyrolysis reaction in an inert solvent having a lower boiling point than diphenylmethane diisocyanate to obtain diphenylmethane diisocyanate.

The inert solvent in the present application refers to a solvent which reacts with neither a reactant nor a product.

In the present application, the solvent having the lower boiling point than the product is selected. When the thermal decomposition reaction occurs, the solvent carrying generated methanol is evaporated out of a system, while a pyrolysis product MDI remains as a heavy component in the solvent. After the reaction, MDI is separated from the solvent so that the product is obtained. On the one hand, the evaporation of the solvent promotes the elimination of methanol and promotes the reaction to proceed forward, thereby improving reaction efficiency. On the other hand, the thermal degradation product MDI remains in the solvent and the solvent is continuously replenished during the reaction, thereby avoiding the polymerization of MDI with a high concentration and improving the yield of MDI.

Meanwhile, under the catalysis of the catalyst, a reaction temperature is reduced and reaction time is shortened so that the pyrolysis reaction is conducted at a relatively low temperature, which greatly reduces energy consumption.

In the present application, the catalyst is selected from an elementary substance and/or an alloy of a metal in Group IB, Group IIB, Group IIIA, Group IVA, Group IVB, Group VB and Group VIII of the periodic table, optionally any one or a combination of at least two of iron, copper, nickel, a copper-aluminum alloy or a copper-nickel alloy, optionally copper and/or the copper-nickel alloy.

Optionally, copper includes any one or a combination of at least two of copper powder, copper foam or nano-copper.

Different from a metal oxide commonly used as the catalyst for diphenylmethane dicarbamate in the prior art, the metal and/or the metal alloy are selected as the catalyst for preparing an aromatic diisocyanate in the present application, which has beneficial effects of a low loss and a high catalytic activity.

Copper foam, copper fiber or copper powder, which has a relatively large specific surface area, can be fully in contact with the reaction raw material, and thus can achieve a better catalytic effect.

Optionally, a mass ratio of the catalyst to diphenylmethane dicarbamate is 1:(5-25), for example, 1:6, 1:9, 1:10, 1:13, 1:15, 1:18, 1:20, 1:21, 1:22, 1:24 or the like, optionally 1:(15-20).

If the catalyst is added in too small an amount, the catalyst cannot achieve a catalytic effect. If the catalyst is added in too large an amount, a polymerization reaction may occur.

Optionally, a mass ratio of diphenylmethane dicarbamate to the inert solvent is 1:(7-50).

If the content of the solvent is too high, a substrate may have too low a concentration, which greatly increases a cost of the process. If the content of the solvent is too low, the substrate has a relatively high concentration and isocyanate is easy to polymerize.

In the present application, the inert solvent is selected from an alkane inert solvent and/or a halogenated hydrocarbon inert solvent; optionally, the inert solvent is any one or a combination of at least two of chlorobenzene, orthodichlorobenzene, o-xylene or p-xylene.

In the present application, the pyrolysis reaction is conducted for 0.1-10 h, for example, 1 h, 2 h, 3 h, 4 h, 5 h, 6 h, 7 h, 8 h, 9 h or the like, optionally 1-5 h, for example, 2 h, 3 h, 4 h or the like.

In the present application, the pyrolysis reaction is conducted at a temperature of 140-280° C., for example, 150° C., 160° C., 170° C., 180° C., 190° C., 200° C., 215° C., 220° C., 230° C., 240° C., 250° C., 260° C., 270° C. or the like, under a pressure of 0.2-1 MPa, for example, 0.3 MPa, 0.4 MPa, 0.5 MPa, 0.6 MPa, 0.7 MPa, 0.8 MPa, 0.9 MPa or the like, optionally 0.2-0.8 MPa.

The pyrolysis reaction of the present application has the advantage of a relatively low reaction temperature so that diphenylmethane diisocyanate with a relatively high yield can be obtained in a relatively short time.

Optionally, diphenylmethane dicarbamate includes any one or a combination of at least two of methyl diphenylmethane dicarbamate, ethyl diphenylmethane dicarbamate, propyl diphenylmethane dicarbamate or butyl diphenylmethane dicarbamate.

Optionally, diphenylmethane diisocyanate includes any one or a combination of at least two of 4,4′-diphenylmethane diisocyanate, 2,4′-diphenylmethane diisocyanate or polymeric diphenylmethane diisocyanate.

Diphenylmethane diisocyanate prepared by the preparation method of the present application includes both 4,4′-MDI and PMDI.

As an optional technical solution, the preparation method includes steps described below.

In the presence of a catalyst, diphenylmethane dicarbamate is subjected to a pyrolysis reaction in an inert solvent having a lower boiling point than diphenylmethane diisocyanate for 0.1-10 h at a temperature of 140-280° C. under a pressure of 0.2-1 MPa to obtain diphenylmethane diisocyanate.

Compared with the prior art, the present application has the beneficial effects below.

(1) In the present application, the solvent having the lower boiling point than the product is selected. When the thermal decomposition reaction occurs, the solvent carrying generated methanol is evaporated out of the system, while the pyrolysis product MDI remains as the heavy component in the solvent. After the reaction, MDI is separated from the solvent so that the product is obtained. On the one hand, the evaporation of the solvent promotes the elimination of methanol and promotes the reaction to proceed forward, thereby improving the reaction efficiency. On the other hand, the thermal degradation product MDI remains in the solvent and the solvent is continuously replenished during the reaction, thereby avoiding the polymerization of MDI with a high concentration and improving the yield of MDI. Meanwhile, under the catalysis of the catalyst, the reaction temperature is reduced and the reaction time is shortened so that the pyrolysis reaction is conducted at a relatively low temperature, which greatly reduces the energy consumption.

(2) The preparation method provided by the present application has the relatively high conversion rate and yield, where the reaction conversion rate can reach 99.9% and the yield can reach up to 98.9%. Moreover, the preparation method has the simple devices and processes and the relatively mild reaction conditions and can meet the requirement for industrial production.

### DETAILED DESCRIPTION

Technical solutions of the present application are further described below through detailed embodiments. Those skilled in the art are to understand that examples described herein are merely used for a better understanding of the present application and are not to be construed as a specific limitation to the present application.

### Example 1

A preparation method for 4,4′-diphenylmethane diisocyanate is described below.

Methyl 4,4′-diphenylmethane dicarbamate, nano-copper and a solvent p-xylene were added to a 1000 mL reaction kettle and then reacted for 2 h at a temperature of 220° C. under a pressure of 0.35 MPa.

A mass ratio of nano-copper to methyl 4,4′-diphenylmethane dicarbamate was 1:20, and a mass ratio of methyl 4,4′-diphenylmethane dicarbamate to the solvent p-xylene was 1:19.

### Examples 2 to 6

Examples 2 to 6 differ from Example 1 only in that nano-copper was added in different amounts and a mass ratio of nano-copper to methyl 4,4′-diphenylmethane dicarbamate was controlled to be 1:5 (Example 2), 1:15 (Example 3), 1:25 (Example 4), 1:4 (Example 5) or 1:30 (Example 6).

### Examples 7 to 10

Examples 7 to 10 differ from Example 1 only in that a solvent p-xylene was added in different amounts and a mass ratio of methyl 4,4′-diphenylmethane dicarbamate to the solvent p-xylene was controlled to be 1:7 (Example 7), 1:50 (Example 8), 1:4 (Example 9) or 1:55 (Example 10).

### Examples 11 and 12

Examples 11 and 12 differ from Example 1 only in that nano-copper was replaced with a copper-nickel alloy (Example 11) or iron (Example 12).

### Example 13

A preparation method for 4,4′-diphenylmethane diisocyanate is described below.

Methyl 4,4′-diphenylmethane dicarbamate, copper fiber and a solvent p-xylene were added to a 1000 mL reaction kettle and then reacted for 2 h at a temperature of 220° C. under a pressure of 0.55 MPa.

A mass ratio of copper fiber to methyl 4,4′-diphenylmethane dicarbamate was 1:19, and a mass ratio of methyl 4,4′-diphenylmethane dicarbamate to the solvent p-xylene was 1:19.

### Example 14

A preparation method for 4,4′-diphenylmethane diisocyanate is described below.

Propyl 4,4′-diphenylmethane dicarbamate, copper foam and a solvent p-xylene were added to a 1000 mL reaction kettle and then reacted for 2 h at a temperature of 250° C. under a pressure of 0.55 MPa.

A mass ratio of copper foam to methyl 4,4′-diphenylmethane dicarbamate was 1:9, and a mass ratio of methyl 4,4′-diphenylmethane dicarbamate to the solvent p-xylene was 1:9.

### Example 15

A preparation method for 4,4′-diphenylmethane diisocyanate is described below.

Butyl 4,4′-diphenylmethane dicarbamate, copper powder and a solvent p-xylene were added to a 1000 mL reaction kettle and then reacted for 2 h at a temperature of 250° C. under a pressure of 0.55 MPa.

A mass ratio of copper foam to methyl 4,4′-diphenylmethane dicarbamate was 1:19, and a mass ratio of methyl 4,4′-diphenylmethane dicarbamate to the solvent p-xylene was 1:19.

### Example 16

A preparation method for 4,4′-diphenylmethane diisocyanate is described below.

Methyl 4,4′-diphenylmethane dicarbamate, copper foam and a solvent o-xylene were added to a 1000 mL reaction kettle and then reacted for 2 h at a temperature of 270° C. under a pressure of 0.55 MPa.

A mass ratio of copper foam to methyl 4,4′-diphenylmethane dicarbamate was 1:19, and a mass ratio of methyl 4,4′-diphenylmethane dicarbamate to the solvent o-xylene was 1:10.

### Example 17

A preparation method for polymeric diphenylmethane diisocyanate is described below.

Polymeric methyl diphenylmethane dicarbamate, copper powder and a solvent chlorobenzene were added to a 1000 mL reaction kettle and then reacted for 2 h at a temperature of 250° C. under a pressure of 0.55 MPa.

A mass ratio of copper foam to polymeric methyl diphenylmethane dicarbamate was 1:20, and a mass ratio of polymeric methyl diphenylmethane dicarbamate to the solvent chlorobenzene was 1:20.

### Example 18

A preparation method for 4,4′-diphenylmethane diisocyanate is described below.

Methyl 4,4′-diphenylmethane dicarbamate, nano-copper and a solvent p-xylene were added to a 1000 mL reaction kettle and then reacted for 0.1 h at a temperature of 280° C. under a pressure of 0.2 MPa.

A mass ratio of nano-copper to methyl 4,4′-diphenylmethane dicarbamate was 1:20, and a mass ratio of methyl 4,4′-diphenylmethane dicarbamate to the solvent p-xylene was 1:19.

### Example 19

A preparation method for 4,4′-diphenylmethane diisocyanate is described below.

Methyl 4,4′-diphenylmethane dicarbamate, nano-copper and a solvent p-xylene were added to a 1000 mL reaction kettle and then reacted for 10 h at a temperature of 140° C. under a pressure of 1 MPa.

A mass ratio of nano-copper to methyl 4,4′-diphenylmethane dicarbamate was 1:20, and a mass ratio of methyl 4,4′-diphenylmethane dicarbamate to the solvent p-xylene was 1:19.

### Comparative Example 1

Comparative Example 1 differs from Example 1 in that nano-copper was replaced with nano-copper(II) oxide as a catalyst.

### Comparative Example 2

Comparative Example 2 differs from Example 1 in that nano-copper was not added as a catalyst.

### Comparative Example 3

Comparative Example 3 differs from Example 1 in that solvent p-xylene was replaced with dioctyl sebacate.

### Performance Test

A performance test was performed on diphenylmethane diisocyanate provided in Examples 1 to 19 and Comparative Examples 1 to 3 by a method described below.

(1) Conversion rate of a reactant: the system was subjected to a chromatographic analysis after its volume was precisely adjusted with methanol/water solution, and a quantitative analysis was performed using an Agilent-1200 high-performance liquid chromatograph developed by Agilent Technologies, Inc. of the United States.

(2) Yield of a product: the system was subjected to the chromatographic analysis after its volume was precisely adjusted with methanol/water solution, and the quantitative analysis was performed using the Agilent-1200 high-performance liquid chromatograph developed by Agilent Technologies, Inc. of the United States.

The results of the test are shown in Table 1.

TABLE 1

| | Conversion rate | Yield of |
|---|---|---|
| Sample | of Reactant/% | Product/% |
| Example 1 | 98.9 | 98.7 |
| Example 2 | 99.2 | 96.5 |
| Example 3 | 99.1 | 97.3 |
| Example 4 | 99.5 | 97.1 |
| Example 5 | 99.6 | 92.3 |
| Example 6 | 98.2 | 92.5 |
| Example 7 | 98.5 | 72.5 |
| Example 8 | 99.2 | 98.4 |
| Example 9 | 98.9 | 50.3 |
| Example 10 | 99.4 | 98.7 |
| Example 11 | 97.4 | 78.2 |
| Example 12 | 96.2 | 72.5 |
| Example 13 | 99.2 | 90.8 |
| Example 14 | 99.9 | 96.6 |
| Example 15 | 98.6 | 92.6 |
| Example 16 | 99.9 | 96.6 |
| Example 17 | 98.5 | 96.2 |
| Example 18 | 40.3 | 15.2 |
| Example 19 | 100 | 54.2 |
| Comparative Example 1 | 92.5 | 67.8 |
| Comparative Example 2 | 91.6 | 48.7 |
| Comparative Example 3 | 90.6 | 75.5 |

From the examples and the performance test, it can be seen that the preparation method for diphenylmethane diisocyanate provided by the present application has the advantages of mild reaction conditions and a relatively high yield, where the conversion rate of the reactant is up to more than 97% and the yield of the product is up to more than 90%.

As can be seen from the comparison of Examples 1 to 6, in the present application, when the mass ratio of the catalyst to the reactant is 1:(5-25), the yield of MDI is relatively high. As can be seen from the comparison of Example 1 with Examples 7 to 10, when the mass ratio of the inert solvent to the reactant is (7-50):1, the yield of MDI is relatively high; when the mass ratio of the inert solvent to the reactant is (19-50):1, the yield of MDI is higher. As can be seen from the comparison of Example 1 with Comparative Example 1, when the metal oxide is selected as the catalyst, the metal oxide has a poorer effect than a metal or a metal alloy. As can be seen from the comparison of Example 1 with Comparative Example 2, when the catalyst is not added, the conversion rate of diphenylmethane dicarbamate is relatively low due to a relatively low reaction temperature and reaction pressure so that the yield of MDI is relatively low. As can be seen from the comparison of Example 1 with Comparative Example 3, when a solvent with a high boiling point is selected, since MDI and methanol are evaporated simultaneously during the reaction, a reversible side reaction is easy to occur and MDI with too high a concentration is easy to polymerize so that the yield of MDI is relatively low.

The applicant states that although the preparation method for diphenylmethane diisocyanate of the present application is described through the preceding examples, the present application is not limited to the preceding examples, which means that implementation of the present application does not necessarily depend on the preceding examples.

1. A preparation method for diphenylmethane diisocyanate, comprising:
in the presence of a catalyst, subjecting diphenylmethane dicarbamate to a pyrolysis reaction in an inert solvent having a lower boiling point than diphenylmethane diisocyanate to obtain diphenylmethane diisocyanate.

2. The preparation method for diphenylmethane diisocyanate according to claim 1, wherein the catalyst is selected from an elementary substance and/or an alloy of a metal in Group IB, Group IIB, Group IIIA, Group IVA, Group IVB, Group VB and Group VIII of the periodic table.

3. The preparation method for diphenylmethane diisocyanate according to claim 1, wherein a mass ratio of the catalyst to diphenylmethane dicarbamate is 1:(5-25).

4. The preparation method for diphenylmethane diisocyanate according to claim 3, wherein the mass ratio of the catalyst to diphenylmethane dicarbamate is 1:(15-20).

5. The preparation method for diphenylmethane diisocyanate according to claim 1, wherein the catalyst is any one or a combination of at least two of iron, copper, nickel, a copper-aluminum alloy or a copper-nickel alloy, optionally copper and/or the copper-nickel alloy; and
optionally, the copper comprises any one or a combination of at least two of copper powder, copper foam or nano-copper.

6. The preparation method for diphenylmethane diisocyanate according to claim 1, wherein a mass ratio of diphenylmethane dicarbamate to the inert solvent is 1:(7-50).

7. The preparation method for diphenylmethane diisocyanate according to claim 1, wherein the inert solvent is selected from an alkane inert solvent and/or a halogenated hydrocarbon inert solvent; optionally, the inert solvent is any one or a combination of at least two of chlorobenzene, orthodichlorobenzene, o-xylene or p-xylene.

8. The preparation method for diphenylmethane diisocyanate according to claim 1, wherein the pyrolysis reaction is conducted for 0.1-10 h, optionally 1-5 h.

9. The preparation method for diphenylmethane diisocyanate according to claim 1, wherein the pyrolysis reaction is conducted at a temperature of 140-280° C. under a pressure of 0.2-1 MPa, optionally 0.2-0.8 MPa.

10. The preparation method for diphenylmethane diisocyanate according to claim 1, wherein diphenylmethane dicarbamate comprises any one or a combination of at least two of methyl diphenylmethane dicarbamate, ethyl diphenylmethane dicarbamate, propyl diphenylmethane dicarbamate or butyl diphenylmethane dicarbamate.

11. The preparation method for diphenylmethane diisocyanate according to claim 1, wherein diphenylmethane diisocyanate comprises any one or a combination of at least two of 4,4′-diphenylmethane diisocyanate, 2,4′-diphenylmethane diisocyanate or polymeric diphenylmethane diisocyanate.

12. The preparation method for diphenylmethane diisocyanate according to claim 1, comprising the following steps:
in the presence of a catalyst, subjecting diphenylmethane dicarbamate to a pyrolysis reaction in an inert solvent having a lower boiling point than diphenylmethane diisocyanate for 0.1-10 h at a temperature of 140-280° C. under a pressure of 0.2-1 MPa to obtain diphenylmethane diisocyanate.
